(19) United States
(12) Patent Application Publication
(10) Pub. No.: US 2009/0238870 A1
(43) Pub. Date: Sep. 24, 2009

(54) Dividable galenical form allowing modified release of the active ingredient

(75) Inventors: Gilles Fonknechten, Ligny Le Ribauld (FR); Patrick Genty, Orleans (FR); Jean-Manuel Pean, Orleans (FR); Patrick Wuthrich, Saint Denis En Val (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, Courbevoie Cedex (FR)

(21) Appl. No.: 12/229,418

(22) Filed: Aug. 22, 2008

(30) Foreign Application Priority Data
Mar. 21, 2008 (FR) 08/01561

(51) Int. Cl. A61K 9/44; A61K 47/38; A61K 31/4035; A61P 3/10

(52) U.S. Cl. 424/467; 514/781; 514/416

(57) ABSTRACT

Dividable galenical form for the modified release of active ingredient, wherein the non-subdivided galenical form and a portion of said form obtained by subdivision have identical dissolution profiles. Medicaments.

The present invention lies within the field of research into and development of new galenical forms of pharmaceutical preparations.

The present invention relates to a dividable galenical form allowing modified release of the active ingredient.

Pharmaceutical compositions having modified release—e.g. prolonged release, delayed release or sequential release—of the active substance have been known for a long time. In particular, they make it possible to avoid peaks of active ingredient in the blood and to obtain a steady blood concentration in humans. This includes reducing the undesirable effects that can occur as a result of the “peak effect”, which may be accompanied by hydroelectrolytic and metabolic-type problems associated with variations in plasma levels of the active ingredient. A modified-release form is especially advantageous, compared to an immediate-release form, in that in certain patients it avoids active ingredient blood concentrations that are elevated and of short duration and the effect of which can prove deleterious in the treatment of certain pathologies.

A dividable galenical form, such as a dividable tablet, has features such as break lines which allow said galenical form to be split and which result in portions of practically equal mass which contain practically equal amounts of the active substance. The subdivision of a tablet constitutes a traditional but nevertheless recurring problem in galenical science. Tablets bearing breaking grooves that allow easy breakage and that make it possible to obtain split doses containing an exact and equal amount of active ingredient are in common use.

The problem of the present invention is to provide one and the same galenical form with conventional and yet mutually antagonistic properties, namely that of being dividable and that of modified release.

The directive CPMP/QWP/604/96 of the EMEA, the European Medicines Agency, explicitly advises against combining the properties of dividability and of prolonged release within one and the same galenical form: “It is bad practice to subdivide prolonged release dosage forms but it may be justified in exceptional cases.”

Although it is the case that tablets having relatively deep breaking grooves allow easier breaking of said tablets and an exact amount of active substance in each split dose, those dividable tablets which have deep breaking grooves and which are used in the form of split doses have a substantial increase in their surface area, corresponding to the break surface, which may reach 20% of the total surface area. This significant increase in surface area in the case of split doses has a highly disruptive effect on the active substance release characteristics. Consequently, in the case of a total surface area that has been considerably increased as a result of the subdivision, the modified release of the active substance from the split doses is modified to the point that said split doses no longer have, or have only in part, the desired properties, in particular linear modified release. Consequently, the use of dividable tablets that have relatively deep breaking grooves gives rise to a lack of certainty in respect of properties and efficacy which is not acceptable for the patient.

In order to remedy the problems associated with the subdivision of modified-release galenical forms, galenical solutions have been envisaged. In particular, a novel form of modified-release dividable tablets has been developed in order that the increase in total surface area due to the break surfaces should be as small as possible on subdivision (FR 2 462 908). That oblong-shaped tablet has precise relative proportions of length/width/height of 2.5 to 5/0.9 to 2/1. In addition, the width constitutes not more than ⅔ of the length, and the total depth of the grooves is adjusted to between ⅓ and ½ of the height so that the product of the area of a break surface and the number of possible fragments constitutes not more than 15% of the external surface area of the non-subdivided tablet. However, because of the increased ease with which those dividable tablets are broken, due to the small area of the break surfaces, said tablets have a tendency to break at the dividing bar, which is a disadvantageous effect in the course of the industrial process.

The problem of the present invention is accordingly to propose an alternative strategy making it possible to circumvent the problems inherent in the development of modified-release dividable tablets already available, with a view to overcoming, at least in part, the disadvantages associated with the subdivision of tablets into split doses. This alternative strategy is based on the originality of the pharmaceutical composition of the galenical form.

The present invention relates to a dividable galenical form, for example a dividable tablet, having modified release and comprising one or more active ingredients and the following excipients: a cellulose derivative polymer and a binder. This new galenical form is characterised in that it has an identical dissolution profile whether it is subdivided or not. For example, the prolonged-release dividable tablet in its non-subdivided form and a portion of said form obtained by subdivision have identical dissolution profiles.

In the context of the invention, “identical dissolution profiles” is understood to mean dissolution kinetics having coefficients of variation without statistically significant differences. The identical in vitro dissolution kinetics according to the invention result in identical plasma kinetics.

The expression “active ingredient” in accordance with the invention includes the active ingredient as such or one of its hydrates, crystalline forms, and addition salts of any of these with a pharmaceutically acceptable acid.

In accordance with the invention, the expression “active substance” or “active ingredient” relates, in non-limiting manner, to the following therapeutic families: antibiotics, cardiovascular agents, analgesics, anti-coagulants, anti-thrombotics, vasoconstrictors, vasodilators, anti-tumour agents, hyperglycaemic and hypoglycaemic agents, anti-inflammatories, anti-arrhythmia agents, anti-cholesterolaemic agents, vitamins, minerals, it being possible for each of those active ingredients to be in association with one another.

Preferably, the active ingredient according to the invention is a hypoglycaemic agent, especially an antidiabetic agent. More preferably, the active ingredient is a sulphonylurea compound.

Preference is given to the active ingredient used in the invention being gliclazide of formula (I):

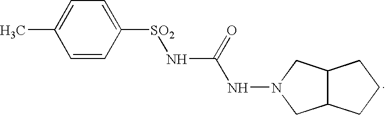

Gliclazide is a sulphonylurea compound recognised for its antidiabetic properties.

The unit dose of gliciazide may vary according to the age and weight of the patient, and the nature and severity of the diabetes. It generally ranges from 30 to 120 mg, in the form of a single administration, for a day's treatment. The percentage of gliclazide within a galenical form is from 12 to 40% of the total weight of the tablet.

Hitherto existing formulations have consisted of:
an immediate-release tablet containing 80 mg; and
a matrix tablet containing 30 mg of gliclazide. This tablet makes it possible to adhere to the unit dose regimen, which ranges from 30 to 120 mg in the form of a single daily administration, corresponding to the taking of from 1 to 4 tablets of 30 mg. This gliclazide tablet, administered in the form of a hydrophilic matrix described in the patent specification EP 1 148 871, makes possible prolonged and controlled release of the active ingredient without the in vitro dissolution kinetics of said matrix being influenced by pH. This form for prolonged release of gliclazide makes it possible to ensure steady plasma levels and low Cₘₐₓ-Cₘᵢₙ variations.

The dosage scheme recommended for gliclazide comprises administering gliclazide at a dose of 30 mg for a first period and then, for a second period, gliclazide at a dose of 60 mg, which is the treatment dose administered to the majority of patients. Furthermore, patients more seriously affected by the disease have to be treated at doses of 90 mg, or even 120 mg, of gliclazide.

In highly advantageous manner compared to existing formulations, the present invention consisting of a dividable prolonged-release matrix tablet containing 60 mg of gliclazide ensures better treatment compliance by limiting the number of tablets to be taken by the patient and also allows manufacture of the medicaments to be optimised on a single production line.

In the formula, the cellulose derivative polymer has the function of forming the matrix, which ensures, inter alia, the modified release of the active ingredient. Release of the active ingredient takes place both by means of diffusion and by means of erosion of the matrix and, in particular, allows prolonged release of the active ingredient.

As understood by the invention, cellulose derivatives, or cellulose polymers, are, for example, ethylcellulose, methylcellulose, cellulose acetate, cellulose acetate phthalate, hydroxymethylcellulose, hydroxyethylcellulose, hydroxypropylcellulose and hydroxypropyl methylcellulose.

Cellulose derivatives that are preferred according to the invention are: hydroxymethylcellulose, hydroxyethylcellulose, hydroxypropylcellulose and hydroxypropyl methylcellulose.

Preference is given to the tablet according to the invention comprising a cellulose derivative of low viscosity. Further preference is given to the tablet according to the invention comprising hydroxypropyl methylcellulose, or HPMC.

HPMCs are commercially available polymers known to the person skilled in the art and customarily used in the field of medicament formulation. It is to be noted that these polymers are marketed especially under the Trade Marks Methocel™ and Metolose™.

A high-viscosity HPMC may be selected from Methocel K15M™ and Methocel K100M™, 2% by weight aqueous solutions of which have viscosities of 15000 and 100000 cP, respectively.

A medium-viscosity HPMC may be selected from Methocel E4M™, Methocel K4M™ and Methocel K4MCR™, 2% by weight aqueous solutions of which have a viscosity of 4000 cP.

A low-viscosity HPMC may be selected from Methocel E5M™, Methocel E5 LV™, Methocel E15 LV™, Methocel E50 LV™, Methocel K100 LV™ and Metolose 90SH100™, 2% by weight aqueous solutions of which have viscosities of 5, 5, 15, 50, 100 and 100 cP, respectively.

In the pharmaceutical composition according to the invention, the binder serves to bind together the particles which cannot be bound together solely by the action of pressure.

The invention relates preferably to binders included in the following list: sucrose solution, glucose solution, sorbitol solution, glucose syrup, preferably maltodextrin, gum arabic, tragacanth, methylcellulose, carboxymethylcellulose, gelatin, starches, PEG 4000 and 6000, polyvidone (PVP) and HPMC of very low viscosity.

The tablet according to the invention preferably comprises maltodextrin, polyvidone or an HPMC of very low molecular weight as binder for the present galenical form.

The present invention accordingly relates preferably to a dividable prolonged-release tablet comprising: a) gliclazide, a cellulose derivative and maltodextrin or b) gliclazide, a cellulose derivative and polyvidone or c) gliclazide, a cellulose derivative and an HPMC of low to very low molecular weight.

In a preferred embodiment, the tablet according to the invention also comprises a hydrophilising agent. In accordance with conventional usage, a hydrophilising agent is understood to be any substance capable of facilitating penetration of the matrix by water so as to rapidly form a gel. In the context of the invention, the hydrophilising agents are those included in the following list: colloidal silica, polysorbate, sorbitol ester. Advantageously, the tablet according to the invention comprises colloidal silica as hydrophilising agent of the present galenical form. The percentage content of colloidal silica as hydrophilising agent in the tablet according to the invention is from 0.1% to 5% of the total weight of the tablet.

The present invention relates especially to a dividable prolonged-release tablet comprising gliclazide, a cellulose derivative, maltodextrin and colloidal silica.

The present invention relates also to a tablet comprising, in addition to the active ingredients and excipients already described,

at least one diluent or filler such as lactose monohydrate, mannitol, a polyol, unsubstituted cellulose or alternatively a starch and a mineral salt, dicalcium phosphate; and /or

at least one lubricant, especially a compression lubricant, such as magnesium stearate or alternatively calcium stearate, zinc stearate, aluminium stearate, sodium stearyl fumarate; and/or

at least one flow agent such as anhydrous colloidal silica.

Preferably, the invention relates to a dividable modified-release tablet comprising from 12% to 40% active ingredient relative to the total weight of the tablet. Preferably, the dividable tablet according to the invention also comprises from 10% to 60% cellulose derivatives relative to the total weight of the tablet. Very especially, the dividable tablet according to the invention comprises from 2% to 15% binder relative to the total weight of the tablet.

Furthermore, the dividable tablet according to the invention bears one or more breaking grooves arranged on one face or on both faces perpendicular to the height and length directions of the tablet. The breaking grooves provided on both faces are preferably opposite one another or else alternate, and furthermore of the same depth or different depths. The dividable tablet can accordingly be split into two or several predetermined portions. This gives rise to its being possible for the dose of the medicament to be matched to the specific dosage regimen associated with the pathology or the patient.

The invention relates preferably to a dividable tablet wherein from 13 to 27% of the total amount of active substance is released within 2 hours, from 32 to 52% of the total amount of active substance is released within 4 hours and more than 85% of the total amount of active substance is released within 12 hours.

Preferably, the tablet according to the invention has the following unitary formula (in mg/tablet) and the following percentage formula:

L0014022:

| | | |
|---|---|---|
| gliclazide | 60.00 | 18.7% |
| lactose monohydrate | 71.36 | 22.3% |
| HPMC 100 cP | 160.00 | 50% |
| maltodextrin | 22.00 | 6.9% |
| anhydrous colloidal silica | 5.04 | 1.6% |
| magnesium stearate | 1.60 | 0.5% |
| Total weight: | 320.00 | |

The following formulation of the tablet according to the invention is given as a function of, on the one hand, the amount in terms of mg/total weight of each compound and, on the other hand, the location of said compound in the internal phase or external phase:

L0014022:

| Internal phase: | |
|---|---|
| Gliclazide | 60 |
| Lactose | 71.36 |
| HPMC 100 cP | 64 |
| Maltodextrin | 22 |
| Anhydrous colloidal silica | 4.4 |
| External phase: | |
| HPMC 100 cP | 96 |
| Magnesium stearate | 1.6 |
| Anhydrous colloidal silica | 0.64 |
| Total weight | 320 |

The invention relates to a process for the preparation, by means of wet granulation, of a dividable tablet as described hereinbefore, comprising at least the following steps:
a) mixing gliclazide, maltodextrin, lactose monohydrate, a portion of the cellulose derivative and a portion of the colloidal silica;
b) after mixing, wetting is carried out; the wet mass thereby obtained is then granulated, dried and classified;
c) the granulate obtained in step b) constitutes an internal phase and is mixed with the remaining portion of the cellulose derivative of low viscosity;
d) lubrication of the granulate obtained in step c), by means of colloidal silica and magnesium stearate;
e) compression of the lubricated mixture using punches which allow breaking grooves to be produced in the tablet.

The invention relates to a process for the preparation, by means of direct compression, of a dividable tablet as described hereinbefore, comprising at least the following steps:
a) mixing gliclazide, maltodextrin, lactose monohydrate, cellulose derivatives and a portion of the colloidal silica;
b) lubrication of the mixture obtained in step a), by means of colloidal silica and magnesium stearate;
c) compression of the lubricated mixture using punches which allow breaking grooves to be produced in the tablet.

Finally, the invention relates to a process for the preparation, by means of compacting granulation or by dry granulation, of a dividable tablet as described hereinbefore, comprising at least the following steps
a) mixing gliclazide, maltodextrin, lactose monohydrate, a portion of the cellulose derivative and a portion of the colloidal silica;
b) after mixing, compacting is carried out and then classifying;
c) the granulate obtained in step b) constitutes an internal phase and is mixed with the remaining portion of the cellulose derivative of low viscosity;
d) lubrication of the granulate obtained in step c), by means of colloidal silica and magnesium stearate;
e) compression of the lubricated mixture using punches which allow breaking grooves to be produced in the tablet.

Preferably, at the end of the process there are obtained tablets whose hardness, measured by crushing across the diameter, is about from 60 to 120 N and the splitting of which by means of the breaking grooves facilitates treatment compliance.

Preferably, the dividable galenical forms for the modified release of gliclazide in accordance with the invention are used in producing medicaments for the treatment of diabetes.

The present invention is illustrated by the following Figures and Examples, without being limited thereby:

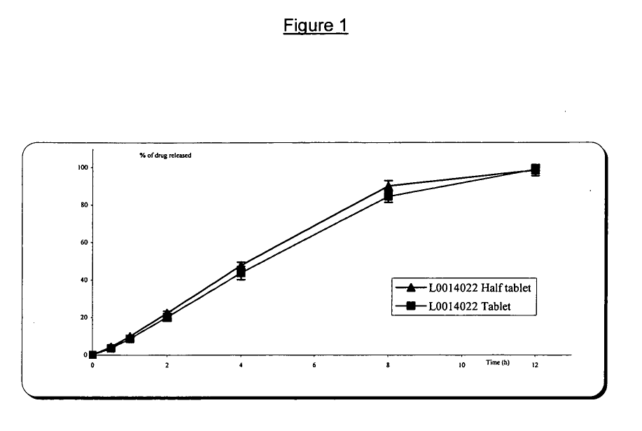

FIG. 1: Comparative dissolution kinetics for a complete tablet and a half tablet of composition L0014022;

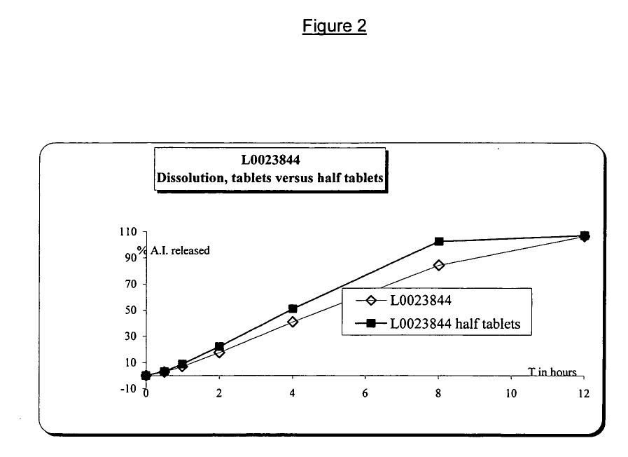

FIG. 2: Comparative dissolution kinetics for a complete tablet and a half tablet of composition L0023844;

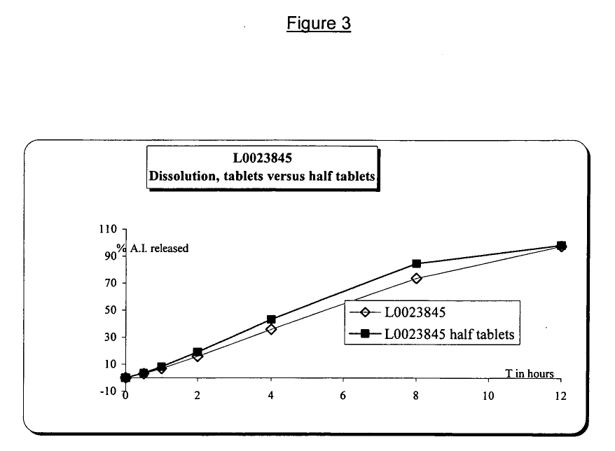

FIG. 3: Comparative dissolution kinetics for a complete tablet and a half tablet of composition L0023845;

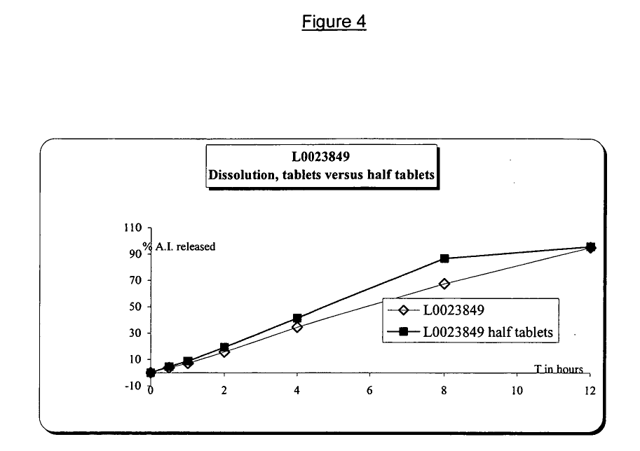

FIG. 4: Comparative dissolution kinetics for a complete tablet and a half tablet of composition L0023849.

### EXAMPLES 1 TO 4

### Comparison of Dissolution Kinetics

Examples 1 to 4 compare the in vitro release kinetics of non-subdivided tablets with the in vitro release kinetics of split doses. The dissolution profiles are compared using the similarity factor (f₂). Two dissolution profiles are considered to be similar when the value of (f₂) is greater than or equal to 50. The directives of the EMEA and the FDA advise calculation of the similarity factor (f₂) in order to compare two dissolution profiles and in order to decide whether said dissolution profiles are identical. The similarity factor (f₂) has the formula:

$${f}_{2} = 50 \cdot log [ \frac{100}{\sqrt{1 + \frac{{\sum }_{r = 1}^{t - n} {[ \overline{R} ( t ) - \overline{T} ( t ) ]}^{2}}{n}}} ]$$

wherein f₂ is the similarity factor, n is the number of standardised points, R(t) is the mean percentage of active ingredient dissolved from the non-subdivided tablet and T(t) is the mean percentage of active ingredient dissolved from a split dose of said tablet. In Examples 1 to 4, the standardised points are at t=2 hours, t=4 hours and t=12 hours.

The tablets assessed have different formulations; those formulations vary especially in the nature of the cellulose derivatives and in the nature of the binders used. The tablets are produced in accordance with the process of the invention described hereinbefore.

### EXAMPLE 1

| | Amount |
|---|---|
| L0014022 | mg |
| Gliclazide | 60 |
| HPMC 100 cP | 64 |
| HPMC 100 cP (external phase) | 96 |
| Povidone | 22 |
| Lactose Monohydrate | 71.36 |
| Anhydrous Colloidal Silica | 0.64 |
| Anhydrous Colloidal Silica (external phase) | 4.4 |
| Magnesium Stearate (external phase) | 1.6 |

| | | | L0014022 | L0014022 | L0014022 |
|---|---|---|---|---|---|
| | L0014022 | L0014022 | Complete | Complete | Complete |
| | ½ tablet | ½ tablet | tablet | tablet | tablet/½ |
| Time | % A.I. | Standard | % A.I. | Standard | tablet |
| (hours) | released | deviation | released | deviation | f₂ |
| 0 | 0 | 0 | 0 | 0 | 75% |
| 0.5 | 4.34 | 0.33 | 3.61 | 0.95 | |
| 1 | 9.93 | 0.57 | 8.68 | 1.05 | |
| 2 | 22.38 | 1.07 | 20.25 | 2.09 | |
| 4 | 47.8 | 1.78 | 43.77 | 3.54 | |
| 8 | 90.17 | 2.87 | 84.64 | 3.26 | |
| 12 | 98.58 | 2.93 | 99.1 | 0.8 | |

### EXAMPLE 2

| L0023844 | mg |
|---|---|
| Gliclazide | 60 |
| HPMC 100 cP | 64 |
| HPMC 100 cP (external phase) | 96 |
| Povidone | 22 |
| Lactose Monohydrate | 71.36 |
| Anhydrous Colloidal Silica | 0.64 |
| Anhydrous Colloidal Silica (external phase) | 4.4 |
| Magnesium Stearate (external phase) | 1.6 |

| | | | L0023844 | L0023844 | L0023844 |
|---|---|---|---|---|---|
| | L0023844 | L0023844 | Complete | Complete | Complete |
| | ½ tablet | ½ tablet | tablet | tablet | tablet/½ |
| Time | % A.I. | Standard | % A.I. | Standard | tablet |
| (hours) | released | deviation | released | deviation | f₂ |
| 0 | 0.00 | 0.00 | 0.00 | 0.00 | 52.8% |
| 0.5 | 3.17 | 0.24 | 2.79 | 0.48 | |
| 1 | 8.94 | 0.44 | 7.04 | 0.59 | |
| 2 | 22.11 | 1.04 | 17.65 | 1.19 | |
| 4 | 51.10 | 1.36 | 41.04 | 1.63 | |
| 8 | 102.52 | 2.42 | 84.31 | 1.52 | |
| 12 | 107.17 | 2.32 | 106.48 | 1.91 | |

### EXAMPLE 3

| L0023845 | mg |
|---|---|
| Gliclazide | 60 |
| HPMC 100 cP | 64 |
| HPMC 100 cP (external phase) | 96 |
| Lactose Monohydrate | 71.36 |
| Anhydrous Colloidal Silica | 0.64 |
| Anhydrous Colloidal Silica (external phase) | 4.4 |
| Magnesium Stearate (external phase) | 1.6 |
| HPMC of low molecular weight | 22 |

| | | | L0023845 | L0023845 | |
|---|---|---|---|---|---|
| | L0023845 | L0023845 | Complete | Complete | L0023845 |
| | ½ tablet | ½ tablet | tablet | tablet | Complete |
| Time | % A.I. | Standard | % A.I. | Standard | tablet/½ tablet |
| (hours) | released | deviation | released | deviation | f₂ |
| 0 | 0.00 | 0.00 | 0.00 | 0.00 | 62.2% |
| 0.5 | 3.53 | 0.68 | 2.97 | 0.05 | |
| 1 | 8.06 | 0.71 | 6.86 | 0.12 | |
| 2 | 19.15 | 0.88 | 15.87 | 0.61 | |
| 4 | 43.17 | 0.94 | 35.74 | 1.14 | |
| 8 | 84.60 | 1.58 | 73.65 | 1.49 | |
| 12 | 98.11 | 2.75 | 97.37 | 2.03 | |

### EXAMPLE 4

| L0023849 | mg |
|---|---|
| Maltodextrin | 22 |
| Hydroxyethycellulose | 64 |
| Hydroxyethycellulose (external phase) | 96 |
| Gliclazide | 60 |
| Lactose Monohydrate | 71.36 |
| Anhydrous Colloidal Silica | 0.64 |
| Anhydrous Colloidal Silica (external phase) | 4.4 |
| Magnesium Stearate (external phase) | 1.6 |

| | | | L0023849 | L0023849 | L0023849 |
|---|---|---|---|---|---|
| | L0023849 | L0023849 | Complete | Complete | Complete |
| | ½ tablet | ½ tablet | tablet | tablet | tablet/½ |
| Time | % A.I. | Standard | % A.I. | Standard | tablet |
| (hours) | released | deviation | released | deviation | f₂ |
| 0 | 0.00 | 0.00 | 0.00 | 0.00 | 53.4% |
| 0.5 | 4.63 | 0.29 | 3.80 | 0.10 | |
| 1 | 8.91 | 0.31 | 7.27 | 0.25 | |
| 2 | 19.32 | 0.62 | 15.78 | 0.58 | |
| 4 | 41.34 | 1.04 | 37.47 | 2.02 | |
| 8 | 86.61 | 2.62 | 67.37 | 3.10 | |
| 12 | 95.71 | 2.76 | 95.00 | 1.92 | |

The pharmaceutical compositions assessed in Examples 1 to 5 have similar dissolution profiles for the non-subdivided form and for the portion of the form obtained by subdivision.

1. A dividable modified-release tablet comprising an active ingredient, a cellulose derivative and a binder, wherein the tablet which has not been subdivided and a portion of the tablet which is obtained by subdividing the tablet have similar dissolution profiles.

2. The tablet of claim 1, wherein the active substance is gliclazide.

3. The tablet of claim 1, wherein the cellulose derivative is selected from hydroxymethylcellulose, hydroxyethylcellulose, hydroxypropylcellulose and hydroxypropyl methylcellulose.

4. The tablet of claim 3, wherein the cellulose derivative is hydroxypropyl methylcellulose of low viscosity.

5. The tablet of claim 1, wherein the binder is maltodextrin, polyvidone or a hydroxypropyl methylcellulose of very low viscosity.

6. The tablet of claim 1, wherein the tablet comprises a hydrophilizing agent.

7. The tablet of claim 6, wherein the hydrophilizing agent is colloidal silica.

8. The tablet of claim 1, which comprises gliclazide, a cellulose derivative, maltodextrin and anhydrous colloidal silica.

9. The tablet of claim 1, wherein the active ingredient comprises from 12% to 40% of the total weight of the tablet.

10. The tablet of claim 1, wherein the cellulose derivative comprises from 10% to 60% of the total weight of the tablet.

11. The tablet of claim 1, wherein the binder comprises from 2% to 15% of the total weight of the tablet.

12. The tablet of claim 2, which comprises 60 mg gliclazide.

13. A dividable modified-release tablet, comprising 18.7% gliclazide, 22.3% lactose monohydrate, 6.9% maltodextrin, 50% low-substituted hydroxypropyl methylcellulose, 0.5% magnesium stearate and 1.6% anhydrous colloidal silica.

14. The tablet of claim 1, which bears one or more breaking grooves perpendicular to the height and length of the tablet.

15. The tablet of claim 1, which exhibits prolonged release.

16. The tablet of claim 1, wherein 13 to 27% of the total amount of active ingredient is released within 2 hours, 32 to 52% of the total amount of active ingredient is released within 4 hours and more than 85% of the total amount of active ingredient is released within 12 hours.

17. A method of treating a living animal body afflicted with diabetes, comprising the step of administering to the living animal body one or more tablets or subdivided tablets of claim 1.
